(19) United States
(12) Patent Application Publication
(10) Pub. No.: US 2015/0018427 A1
(43) Pub. Date: Jan. 15, 2015

(54) COMPOUNDS FOR TREATING PARVOVIRUS INFECTION

(71) Applicant: ARATANA THERAPEUTICS NV, HeverIee (BE)

(72) Inventors: Nesya Goris, Kessel-Lo (BE); Johan Neyts, Kessel-Lo (BE); Erwin Blomsma, Linden (BE); Stefaan Wera, Bierbeek (BE); Jérôme Villers, Roux (BE); Ainola Billiet, Lovenjoel (BE); Joeri Auwerx, Bierbeek (BE); Veerle Debeurme, Kessel-Lo (BE); Eleonora Kiss, Leuven (BE); Chloë Swinnen, Haasrode (BE)

(73) Assignee: Aratana Therapeutics NV, Hererlee (BE)

(21) Appl. No.: 14/380,515

(22) PCT Filed: Feb. 22, 2013
(86) PCT No.: PCT/EP2013/053526

§ 371 Date: Aug. 22, 2014

(30) Foreign Application Priority Data
Feb. 24, 2012 (GB) 1203180.3

(51) Int. Cl. C07C 211/38; A61K 31/13

(52) U.S. Cl. CPC C07C 211/38; A61K 31/13
USPC 514/662

(57) ABSTRACT

The present invention relates to compounds for use in a method for the treatment or prevention of parvovirus infections in humans and warm-blooded animals, including feline panleukopenia virus (FPV) infections in felids, canine parvovirus type 2 (CPV-2) infections in canines, minute virus of mice (MVM) infections in mice and B19 parvovirus infections in humans.

### FIELD OF THE INVENTION

The present invention relates to compounds for use in a method for the treatment or prevention of parvovirus infections in humans and warm-blooded animals, including feline panleukopenia virus (FPV) infections in felids, canine parvovirus type 2 (CPV-2) infections in canines, Minute Virus of Mice (MVM) infections in mice and B19 parvovirus infections in humans.

### BACKGROUND OF THE INVENTION

Parvoviruses (family Parvoviridae) are small, non-enveloped, single-stranded DNA viruses that are mostly species specific. Parvoviridae are divided in the subfamilies of Densovirinae, infecting only invertebrates, and Parvovirinae, infecting vertebrates. The Parvovirinae subfamily is divided in the genera of Amdovirus, Bocavirus, Dependovirus, Erythrovirus and Parvovirus. In humans, human parvovirus B19 is the only member of the Parvoviridae known to be pathogenic. This B19 parvovirus is a member of the erythroviruses and is common and widespread. Manifestations of a B19 infection vary with the immunologic and hematologic status of the host. B19 exclusively infects humans and shows a pronounced tropism for erythroid precursors. B19 uses at least 3 cellular (co)-receptors for attachment and entry i.e. glycolipid globoside or group P antigen, α5δ1-integrin and Ku80 auto-antigen.

The prevalence of IgG antibodies directed against human parvovirus B19 increases with age and ranges from 2-15% in children of 1-5 years old, to 15-60% in children 6-19 years old, to 30-60% in adults, and to more than 85% in geriatric population. Women of childbearing age show an annual seroconversion rate of 1.5%. Although antibodies are prevalent in the general population, viremia or presence of viral DNA is rare. The frequency of B19 viremia in voluntary blood donors has been estimated at rates of 1:167 to 1:35,000. Transmission of infection occurs via the respiratory route or through blood-derived products administered parenterally or vertically from mother to fetus. The spectrum of disease linked to B19 primarily involves infection in the healthy host manifested as:
Erythema infectiosum, also called slapped cheek disease or fifth disease and the most prevalent manifestation of B19 in children;
Athralgia and arthritis in adults (30-60% of human parvovirus B19 infected persons) and less frequently in children;
Red cell aplasia and hemolysis due to the affinity of B19 for the erythroid system;
Hydrops fetalis and spontaneous abortion as a cause of fetal anemia. Fetal death occurs in 10% of cases when infection occurs during the first 20 weeks of pregnancy.

Current treatment of human parvovirus B19 depends on severity and manifestation of the infection and status of the patients and consists of nonsteroidal anti-inflammatory drugs, (fetal) blood transfusions or intravenous immunoglobulin administration. However, infection in healthy patients is often self-limiting and requires no specific agents. In immunocompromised patients, B19 infection may become persistent.

In cats and dogs, parvovirus replication occurs only in rapidly dividing cells such as intestinal crypt epithelial cells, precursor cells in the bone marrow and myocardiocytes. Viral replication results in cell death and loss due to failure of mitosis.

Canine parvovirus type 2 (CPV-2) was the second parvovirus described in dogs in 1978 after canine minute virus (CPV-1) in 1967, which is a member of the genus Bocavirus. CPV-2 has 3 antigenic variants designated CPV-2a, CPV-2b and CPV-2c. CPV-2 emerged as a dog pathogen likely through adaptation of a feline panleukopenia (FPV)-like parvovirus of wild carnivores. There are at least six amino acids changes between FPV and CPV-2, mostly accumulated in the capsid protein gene (VP2 domain) that interacts with the host-cell transferrin receptor. CPV-2 variants are all able to infect cats and may even cause disease. In dog shelters and dog hospitals the prevalence of antibodies against CPV-2 can be as high as 58 to 67%.

The most characteristic clinical form induced by CPV-2 is represented by acute enteritis in puppies up to 6 months of age. Clinical signs occur after an incubation period of 3-7 days and consist of anorexia, depression, vomiting, mucoid or bloody diarrhea, dehydration and fever. Subclinical and unapparent infections as well as leukopenia are frequently observed. Mortality rates can be high in pups (70% and more) but are usually less than 1% in adult dogs. Dogs infected with CPV-2 usually die from dehydration or secondary infection rather than the virus itself. CPV-2 spreads rapidly among dogs via fecal-oral route or through oronasal exposure to fomites contaminated by feces. Fecal excretion of the virus was detected as early as 3 days after infection and virus shedding may continue for a maximum period of 4 weeks after clinical or subclinical disease.

A number of CPV-2 attenuated vaccines of canine origin are available in Europe and are licensed for administration to 4-12 week old puppies. In older animals, it is the common practice to give an annual booster vaccination. Complete cross-protection has been reported between all 3 antigenic variants. Data show that 93.7% of vaccinated dogs showed adequate antibody response more than 2 years following vaccination. Because no agent-specific treatment exists for CPV-2 enteritis, management of disease is limited to supportive care and requires hospitalization and aggressive treatment with crystalloid fluids, synthetic and natural colloids, correction of hypoglycemia and electrolyte disturbances, combination antimicrobials, antiemetics, analgesics, enteral nutritional support and anthelmintics. Lately, the use of recombinant human granulocyte colony stimulating factor (G-CSF) was investigated but no benefit in treated puppies was demonstrated. Interferons (IFN) have the ability to modulate several cellular and immune functions, as well as affect virus replication. Despite the lack of canine IFN products, several studies have shown recombinant feline IFN-omega to significantly ameliorate severe enteritis caused by CPV-2 and reduce mortality. The benefit of oseltamivir, an antiviral drug that inhibits neuraminidase (NA), as a therapeutic agent in CPV-2 was investigated recently. However, CPV-2 does not rely on NA for replication and the study showed no significant improvement in the CPV-2 patients (Savigny et al., J. Vet. Emerg. Crit. Care (San Antonio) 2010, 20, 132-142).

FPV is known to infect, besides cats, also other members of the family Felidae, as well as raccoons, minks and foxes. FPV replication in dogs was seen only in some lymphoid tissues but not in the gut and virus was not shed. FPV causes a systemic infection in cats and the virus is transmitted fecal-orally (like CPV-2) and initially replicates in tissues of the oropharynx and is then distributed via a cell-free viremia to virtually all tissues. FPV infects lymphoid tissues and through cellular depletion can cause functional immunosuppression and lymphopenia. The bone marrow is affected as well and the virus has dramatic effect on virtually all myeloid cell populations. This is also reflected by the panleukopenia observed in FPV infected cats. Like for CPV-2, the hallmark of FPV infection is diarrhea. Intra-uterine or perinatal infection may affect the central nervous system of the fetus leading to cerebellar ataxia and intention tremor in affected kittens. Although FPV affects cats of all ages, kittens are most susceptible with a mortality rate as high as 90% and more.

Several inactivated FPV vaccines are available for administration (at 8-9 weeks of age) and provide solid immunity against the disease. A booster vaccine is recommended 1 year after the kitten vaccination course and with intervals of 3 years. However it was shown that cats having responded to FPV vaccination maintained a solid immunity to FPV for at least 7 years in the absence of booster vaccinations or natural challenge. Disease management consists mainly of supportive therapy and good nursing mostly including restoration of fluid and electrolytes. Antibiotics, anti-emetics, vitamin complexes and eventual plasma or whole blood transfusion and parenteral nutrition may be needed in anorexic cats. Feline recombinant IFN-omega inhibits FPV replication in cell culture but so far no data are available on its efficacy in FPV infected cats.

Until today small molecules demonstrating significant activity against FPV or CPV-2 or human parvovirus B19 have not been identified. An improved therapy against parvovirus infections based on small molecule inhibitors would be of high importance in order to treat, in an effective and cost-efficient way, humans suffering from B19 infections as well as companion animals or other felines and canines that suffer from FPV and/or CPV-2.

Amantadine is indicated for various conditions that can be treated by NMDA receptor antagonists including the treatment of idiopathic Parkinson's disease (Paralysis Agitans), postencephalitic Parkinsonism, and symptomatic Parkinsonism which may follow injury to the nervous system by carbon monoxide intoxication. Amantadine is also used for the prophylaxis and treatment of influenza A virus. However, the use of amantadine for the treatment or prevention of parvovirus infection has not been suggested in the art.

### SUMMARY OF THE INVENTION

In a first aspect, the present invention relates to the use of a compound of the amantadine family for the treatment or prevention of a parvovirus infection in a human or animal. Thus, according to one aspect the invention provides a compound of the amantadine family, or a solvate, hydrate, pharmaceutically acceptable salt or veterinary acceptable salt thereof, or a composition comprising one or more of such compounds for use in a method for the treatment or prevention of a parvovirus infection in a human or a warm-blooded animal.

More particularly, the compound of the amantadine family is a compound of formula (I), or a stereoisomer, salt, solvate or hydrate thereof

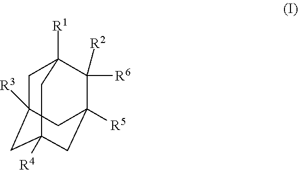

wherein R¹ is selected from amino, aminoC₁₋₆alkyl, C₁₋₆alkylamino and C₁₋₆alkylaminoC₁₋₆alkyl, and R² and R⁶ are hydrogen; or wherein R¹ is hydrogen, R² is selected from amino, aminoC₁₋₆alkyl or C₁₋₆alkylamino, and R⁶ is hydrogen or C₁₋₆alkyl; and
R³, R⁴ and R⁵ are each independently selected from hydrogen, C₁₋₆alkyl and hydroxyl; for use in a method for the treatment or prevention of a parvovirus infection in a human or a warm-blooded animal.

In further embodiments, R¹ is amino and R² and R⁶ are hydrogen; or R¹ is hydrogen, R² is selected from amino, aminoC₁₋₆alkyl or C₁₋₆alkylamino, and R⁶ is hydrogen or C₁₋₆alkyl; and R³, R⁴ and R⁵ are hydrogen or C₁₋₆alkyl. In yet further embodiments, R² is amino.

In certain embodiments, R⁶ is hydrogen. In further embodiments, the compound of the amantadine family is a compound of formula (Ia), or a stereoisomer, salt, solvate or hydrate thereof;

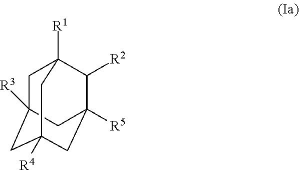

wherein R¹ is amino, and R² is hydrogen; or wherein R¹ is hydrogen and R² is selected from amino, aminoC₁₋₆alkyl or C₁₋₆alkylamino; and R³, R⁴ and R⁵ are each independently selected from hydrogen and C₁₋₆alkyl. In yet further embodiments, R² is amino.

In particular embodiments, said compound is selected from amantadine, memantine, 2-adamantylamine, 2-methyladamantan-2-amine, and 2-ethyladamantan-2-amine, or a solvate, hydrate, pharmaceutically acceptable salt or veterinary acceptable salt thereof. In certain embodiments, the compound is selected from amantadine, memantine, and 2-adamantylamine, or a solvate, hydrate, pharmaceutically acceptable salt or veterinary acceptable salt thereof. In particular embodiments, the compound is amantadine or amantadine hydrochloride. In certain embodiments, the compound is memantine.

In certain embodiments, the compound is for use in a method for lowering the viral load of said parvovirus. In particular embodiments, said parvovirus infection is caused by a virus of the subfamily Parvovirinae. In certain embodiments, the compound is for use in a method for the treatment or prevention of a human parvovirus B19 infection in a human.

In particular embodiments, said warm-blooded animal is a canine. In further embodiments, the compound is for use in a method for the treatment or prevention of a canine parvovirus infection in a canine.

In certain embodiments, said warm-blooded animal is a felid. In further embodiments, the compound is for use in a method for the treatment or prevention of a feline panleukopenia virus infection in a felid.

In a further aspect of the invention, a pharmaceutical composition is provided, said composition comprising one or more compounds of formula (I), or a solvate, pharmaceutically acceptable salt or veterinary acceptable salt thereof;

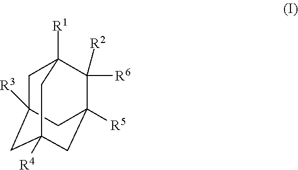

wherein R¹ is selected from amino, aminoC₁₋₆alkyl, C₁₋₆alkylamino and C₁₋₆alkylaminoC₁₋₆alkyl, and R² and R⁶ are hydrogen; or wherein R¹ is hydrogen, R² is selected from amino, aminoC₁₋₆alkyl or C₁₋₆alkylamino, and R⁶ is hydrogen or C₁₋₆alkyl; and
R³, R⁴ and R⁵ are each independently selected from hydrogen, C₁₋₆alkyl and hydroxyl; for use in a method for the treatment or prevention of a parvovirus infection in a human or a warm-blooded animal.

### BRIEF DESCRIPTION OF THE DRAWINGS

The following description of the figures of specific embodiments of the invention is merely exemplary in nature and is not intended to limit the present teachings, their application or uses.

FIG. 1 Structure formula of various compounds of the amantadine family.

FIG. 2 Structure formula of salts of various compounds of the amantadine family.

### DETAILED DESCRIPTION OF THE INVENTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited components, elements or method steps also include embodiments which “consist of” said recited components, elements or method steps.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The values as used herein when referring to a measurable value such as a parameter, an amount, a temporal duration, and the like, is meant to encompass variations of +/−20%, +/−15%, +/−10% or less, preferably +/−5% or less, more preferably +/−1% or less, and still more preferably +/−0.1% or less of and from the specified value, insofar such variations are appropriate to perform in the disclosed invention. It is to be understood that each value as used herein is itself also specifically, and preferably, disclosed.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

The term “parvovirus” as used herein refers to a virus which is a member of the family Parvoviridae, preferably from the subfamily Parvoririnae. Exemplary parvoviruses include, but are not limited to, feline panleukopenia virus, canine parvovirus type 2, human parvovirus B19, minute virus of mice, bovine parvovirus, canine parvovirus, chicken parvovirus and goose parvovirus.

As used herein, the term “puppy” refers to a (domestic) dog which is about 3 years old or less, alternately about 2 years old or less, alternately about 1 year old or less,

As used herein, the term “kitten” refers to a (domestic) cat which is about 3 years old or less, alternately about 2 years old or less, alternately about 1 year old or less.

As used herein, the term “child” refers to a human between the stages of birth and puberty, preferably a human about 12 years or less.

The term “warm-blooded animal” as used herein includes companion animals, such as dogs and cats; domestic animals, such as horses, cattle, sheep, swine, goats, rabbits and chickens; and laboratory animals such as mice, rats and monkeys. Preferably, the warm-blooded animal is mammal, more preferably a companion animal such as a cat or a dog.

The term “alkyl” by itself or as part of another substituent, refers to a straight or branched saturated hydrocarbon group joined by single carbon-carbon bonds having 1 to 6 carbon atoms, for example 1 to 5 carbon atoms, for example 1 to 4 carbon atoms, preferably 1 to 3 carbon atoms. Alkyl groups may be linear or branched and may be substituted as indicated herein. When a subscript is used herein following a carbon atom, the subscript refers to the number of carbon atoms that the named group may contain. Thus, for example, C₁₋₆alkyl means an alkyl of one to six carbon atoms. Examples of C₁₋₄alkyl groups are methyl, ethyl, propyl, isopropyl, butyl, isobutyl and tert-butyl.

As used herein, the term “C₁₋₆alkylene”, by itself or as part of another substituent, refers to C₁₋₆alkyl groups that are divalent, i.e., with two single bonds for attachment to two other groups. Alkylene groups may be linear or branched and may be substituted as indicated herein. Non-limiting examples of alkylene groups include methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and its chain isomers, hexylene and its chain isomers.

The term “amino” by itself or as part of another substituent, refers to —NH₂.

The term “aminoC₁₋₆alkyl”, by itself or as part of another substituent, refers to a group of formula —Rⁱ—NH₂ wherein R′ is C₁₋₆alkylene as defined herein.

The term “C₁₋₆alkylaminoC₁₋₆alkyl”, by itself or as part of another substituent, refers to a group of formula —Rⁱ—NR^{k}R^{l} wherein R^{j} is C₁₋₆alkylene as defined herein, R^{k} is hydrogen or C₁₋₆alkyl as defined herein, and R′ is C₁₋₆alkyl as defined herein.

The term “C₁₋₆alkylamino”, by itself or as part of another substituent, refers to the group —NR^{m}Rⁿ wherein R^{m} is C₁₋₆alkyl as defined herein and Rⁿ is hydrogen or C₁₋₆alkyl as defined herein.

The compounds of formula (I) may have one or more centers of chirality and may exist as stereochemically isomeric forms. The term “stereoisomer” as used herein defines all the possible compounds made up of the same atoms bonded by the same sequence of bonds but having different three-dimensional structures, which the compounds of formula (I) may possess.

The term “pharmaceutically acceptable salts” or “veterinary acceptable salts” as used herein means the therapeutically active non-toxic addition salt forms which the compounds of formula are able to form and which may conveniently be obtained by treating the base form of such compounds with an appropriate base or acid. The pharmaceutically acceptable acid and base addition salts as mentioned herein are meant to comprise the therapeutically active non-toxic acid and base addition salt forms which the compounds according to the present invention are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The term “solvate” is used herein to describe a molecular complex comprising a particular compound and a stoichiometric amount of one or more pharmaceutically acceptable solvent molecules, for example, ethanol. The term “hydrate” is employed when said solvent is water.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the features of the claimed embodiments can be used in any combination.

The inventors have found that amantadine and compounds related to amantadine show a surprisingly strong inhibition of parvovirus replication. Specifically, it was found that compounds of the amantadine family such as amantadine, memantine, 2-adamantylamine, 2-methyladamantan-2-amine, and 2-ethyladamantan-2-amine inhibit replication of human parvovirus B19, feline panleukopenia virus (FPV), canine parvovirus type 2 (CPV-2) and minute virus of mice (MVM). Therefore, these compounds are useful for the treatment or prevention of parvovirus infections in humans and animals. More particularly, these compounds are useful for lowering the parvovirus viral load in humans and animals.

In some embodiments, compounds of the amantadine family are typically compounds of formula (I), stereoisomers, salts, solvates or hydrates thereof;

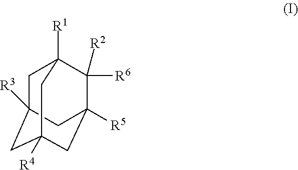

wherein R¹ is selected from amino, aminoC₁₋₆alkyl, C₁₋₆alkylamino and C₁₋₆alkylaminoC₁₋₆alkyl, and R² and R⁶ are hydrogen; preferably R¹ is selected from amino, aminoC₁₋₄alkyl, C₁₋₄alkylamino and C₁₋₄alkylaminoC₁₋₄alkyl; preferably R¹ is selected from amino, aminoC₁₋₃alkyl, C₁₋₃alkylamino and C₁₋₃alkylaminoC₁₋₃alkyl; preferably R¹ is selected from amino, aminomethyl, 1-amino-1-methyl-methyl, or methylamino; more preferably R¹ is selected from amino, aminomethyl, or methylamino; or
wherein R¹ is hydrogen, R² is selected from amino, aminoC₁₋₆alkyl or C₁₋₆alkylamino, and R⁶ is hydrogen or C₁₋₆alkyl; preferably R² is selected from amino, aminoC₁₋₄alkyl, C₁₋₄alkylamino and C₁₋₄alkylaminoC₁₋₄alkyl; preferably R² is selected from amino, aminoC₁₋₃alkyl, C₁₋₃alkylamino and C₁₋₃alkylaminoC₁₋₃alkyl; preferably R² is selected from amino, aminomethyl, 1-amino-1-methyl-methyl, or methylamino; more preferably R² is selected from amino, aminomethyl, or methylamino; most preferably R² is amino
and
R³, R⁴ and R⁵ are each independently selected from hydrogen, C₁₋₆alkyl and hydroxyl; preferably R³, R⁴ and R⁵ are each independently selected from hydrogen, C₁₋₄alkyl and hydroxyl, for example R³, R⁴ and R⁵ are each independently selected from hydrogen, C₁₋₃alkyl and hydroxyl, for example R³, R⁴ and R⁵ are each independently selected from hydrogen, methyl, ethyl, isopropyl and hydroxyl.

In some embodiments, R¹, R² and R⁶ have the same meaning as that defined above, R³ is selected from hydrogen, C₁₋₆alkyl or hydroxyl, preferably from hydrogen, C₁₋₄alkyl and hydroxyl, for example from hydrogen, methyl and hydroxyl, and R⁴ and R⁵ are independently selected from hydrogen or C₁₋₆alkyl, preferably from hydrogen, or C₁₋₄alkyl.

In particular embodiments, R² and R⁶ are hydrogen.

In certain embodiments, R³, R⁴ and R⁵ are each independently selected from hydrogen and C₁₋₆alkyl. In certain embodiments, R³, R⁴ and R⁵ are each independently selected from hydrogen and C₁₋₄alkyl, for example R³, R⁴ and R⁵ are each independently hydrogen or methyl.

In particular embodiments, R¹ is amino and R² and R⁶ are hydrogen; or R¹ is hydrogen, R² is selected from amino, aminoC₁₋₆alkyl or C₁₋₆alkylamino, and R⁶ is hydrogen or C₁₋₆alkyl; and R³, R⁴ and R⁵ are each independently selected from hydrogen and C₁₋₆alkyl. In certain embodiments, R¹ is amino and R² and R⁶ are hydrogen; or R¹ is hydrogen, R² is amino, and R⁶ is hydrogen or C₁₋₆alkyl; and R³, R⁴ and R⁵ are each independently selected from hydrogen and C₁₋₆alkyl.

In some embodiments R¹ is amino, R² and R⁶ are hydrogen; and R³, R⁴ and R⁵ are each independently hydrogen or C₁₋₆alkyl, for example hydrogen or methyl.

In some embodiments R¹ is hydrogen, R² is amino, R⁶ is hydrogen or C₁₋₆alkyl, and R³, R⁴ and R⁵ are each independently hydrogen or C₁₋₆alkyl, for example hydrogen or methyl. If R⁶ is C₁₋₆alkyl, R⁶ preferably is C₁₋₄alkyl, more preferably C₁₋₃alkyl, and most preferably methyl or ethyl.

In particular embodiments R⁶ is hydrogen. Accordingly, in these embodiments, the compounds of the amantadine family are compounds of formula (Ia)

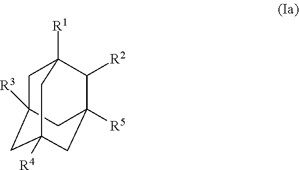

wherein R¹, R², R³, R⁴ and R⁵ have the same meaning as that defined above.

Methods of synthesis of compounds of the amantadine family are know in the art such as described in U.S. Pat. No. 3,310,469 and U.S. Pat. No. 5,599,998, which are hereby incorporated by reference. The amantadine salt 1-Aminoadamantane hydrochloride (amantadine hydrochloride) is sold under the name Symmetrel®. Another member of the amantadine family is rimantadine, which is used in the treatment and prevention of influenza A and sold under the name Flumadine®.

Accordingly, in a first aspect, the present invention relates to the use of compounds of the amantadine family and compositions comprising such compounds in the prevention and treatment of a parvovirus infection in a human or warm-blooded animal.

Thus the invention provides a compound from the amantadine family for use in a method for the treatment or prevention of a parvovirus infection in a human or a warm-blooded animal as well as well as methods for the treatment and/or prevention of a parvovirus infection involving the administration of said compounds. In particular embodiments, the compound of the amantadine family envisaged in the context of the present invention is selected from amantadine, rimantadine, memantine, 3-amino-1-amantadol, 1-adamantanemethylamine, N-methyl-1-adamantylamine, 2-adamantylamine, 2-methyladamantan-2-amine, 2-ethyladamantan-2-amine, or solvates, pharmaceutically acceptable salts or veterinary acceptable salts thereof.

The inventors have further found that certain subsets of compounds are particularly active against parvovirus infection. In certain embodiments, the compound of the amantadine family envisaged in the context of the present invention is selected from amantadine, memantine, 3-amino-1-amantadol, 1-adamantanemethylamine, N-methyl-1-adamantylamine, 2-adamantylamine, 2-methyladamantan-2-amine, and 2-ethyladamantan-2-amine, or solvates, pharmaceutically acceptable salts or veterinary acceptable salts thereof. In further embodiments, the compound of the amantadine family envisaged in the context of the present invention is selected from amantadine, memantine, 3-amino-1-amantadol, 1-adamantanemethylamine, N-methyl-1-adamantylamine and 2-adamantylamine, or solvates, pharmaceutically acceptable salts or veterinary acceptable salts thereof.

In certain embodiments, the compound of the amantadine family envisaged in the context of the present invention is selected from amantadine, memantine and 2-adamantylamine, 2-methyladamantan-2-amine, and 2-ethyladamantan-2-amine, or solvates, pharmaceutically acceptable salts or veterinary acceptable salts thereof. In further embodiments, the compound of the amantadine family is selected from amantadine, memantine and 2-adamantylamine, or solvates, pharmaceutically acceptable salts or veterinary acceptable salts thereof.

In certain embodiments, the compound of the amantadine family is 2-methyladamantan-2-amine or 2-ethyladamantan-2-amine, or solvates, pharmaceutically acceptable salts or veterinary acceptable salts thereof.

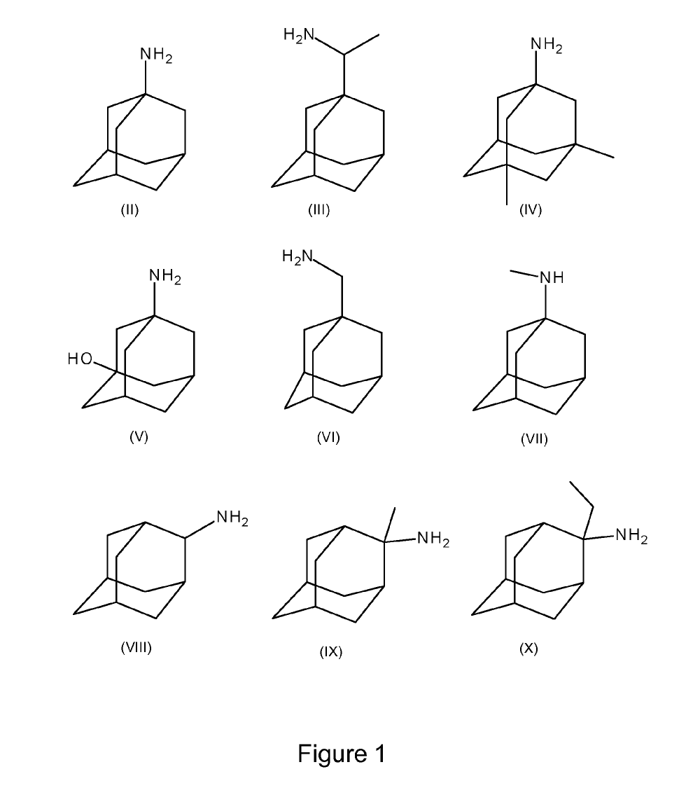

The compounds amantadine, rimantadine, memantine, 3-amino-1-amantadol, 1-adamantanemethylamine, N-methyl-1-adamantylamine, 2-adamantylamine, 2-methyladamantan-2-amine and 2-ethyladamantan-2-amine are represented by formula (II), (III), (IV), (V), (VI), (VII), (VIII), (IX) and (X), respectively (FIG. 1).

In particular embodiments, the compound envisaged for the treatment of a parvovirus infection according to the present invention is amantadine or a solvate or pharmaceutically or veterinary acceptable salt thereof. In certain embodiments, the compound is rimantadine or a pharmaceutically acceptable salt thereof. In certain embodiments, the invention provides the use of is memantine or a solvate or pharmaceutically or veterinary acceptable salt thereof. In certain embodiments, the invention provides the use 3-amino-1-amantadol or a solvate or pharmaceutically or veterinary acceptable salt thereof for the treatment or prevention of a parvovirus infection. In certain embodiments, the compound is 1-adamantanemethylamine or a solvate or pharmaceutically or veterinary acceptable salt thereof. In certain embodiments, the compound is N-methyl-1-adamantylamine or a solvate or pharmaceutically or veterinary acceptable salt thereof. In certain embodiments, the compound is 2-adamantylamine or a solvate or pharmaceutically or veterinary acceptable salt thereof. In certain embodiments, the compound is 2-methyladamantan-2-amine or a solvate or pharmaceutically or veterinary acceptable salt thereof. In certain embodiments, the compound is 2-ethyladamantan-2-amine or a solvate or pharmaceutically or veterinary acceptable salt thereof.

In certain embodiments, the pharmaceutically (or veterinary) acceptable salt of the compounds envisaged for use in the methods of the present invention is an acid addition salt. In further embodiments, the acid addition salt is a hydrochloride or a sulphate.

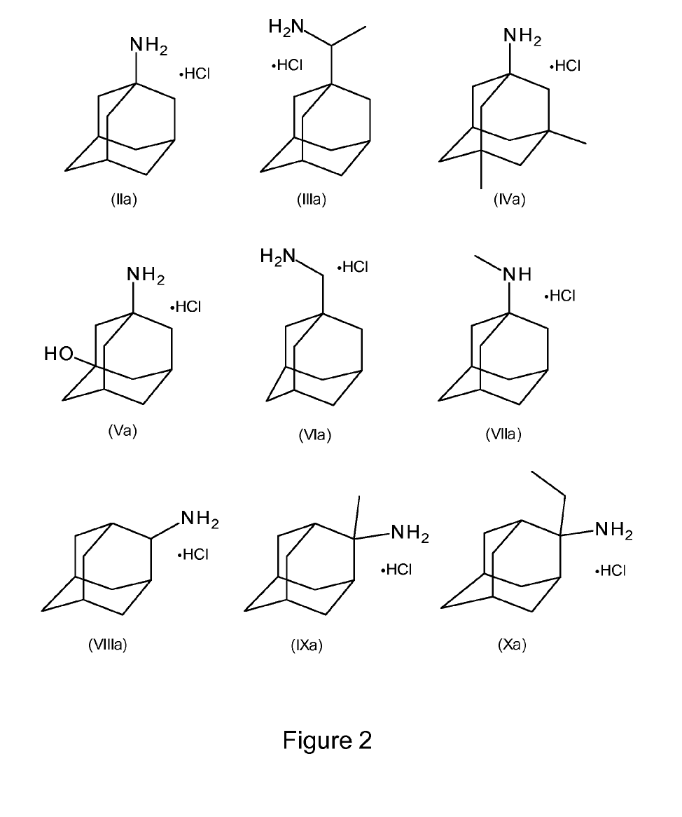

Hydrochlorides may be prepared by mixing the compound according to the present invention with hydrochloric acid. Conversion of the compounds according to the present invention into acid addition salts typically increases the solubility of the compounds in water, and facilitates release of the compound in the gastrointestinal tract. Furthermore, conversion into an acid addition salt may prolong the shelf-life of the compounds envisaged for use in the compositions according to the invention. Accordingly, in particular embodiments, the pharmaceutically (or veterinary) acceptable salt of the compounds envisaged in the context of the present invention is selected from amantadine hydrochloride, rimantadine hydrochloride, memantine hydrochloride, 3-amino-1-amantadol hydrochloride, 1-adamantanemethylamine hydrochloride, N-methyl-1-adamantylamine hydrochloride, 2-adamantylamine hydrochloride, 2-methyladamantan-2-amine hydrochloride, and 2-ethyladamantan-2-amine hydrochloride, which are represented by formula (IIa), (IIIa), (IVa), (IVa), (Va), (Via), (Vila), (Villa), (IXa) and (Xa), respectively (FIG. 2).

In particular embodiments, the pharmaceutically (or veterinary) acceptable salt of the compounds envisaged in the context of the present invention is selected from amantadine hydrochloride, memantine hydrochloride, 3-amino-1-amantadol hydrochloride, 1-adamantanemethylamine hydrochloride, N-methyl-1-adamantylamine hydrochloride and 2-adamantylamine hydrochloride, 2-methyladamantan-2-amine hydrochloride, and 2-ethyladamantan-2-amine hydrochloride. In certain embodiments, the pharmaceutically (or veterinary) acceptable salt is selected from amantadine hydrochloride, memantine hydrochloride, 2-adamantylamine hydrochloride, 2-methyladamantan-2-amine hydrochloride, and 2-ethyladamantan-2-amine hydrochloride. In further embodiments, the pharmaceutically (or veterinary) acceptable salt is selected from amantadine hydrochloride, memantine hydrochloride and 2-adamantylamine hydrochloride.

In certain embodiments, the compound envisaged for the treatment or prevention of a parvovirus infection according to the present invention is amantadine, amantadine hydrochloride, rimantadine, memantine, 3-amino-1-amantadol, 1-adamantanemethylamine, N-methyl-1-adamantylamine or 2-adamantylamine HCl, for example amantadine, amantadine hydrochloride, memantine or 2-adamantylamine HCl.

In particular embodiments, the compound according to the present invention is amantadine hydrochloride.

In particular embodiments, the pharmaceutically (or veterinary) acceptable salt of the compounds envisaged in the context of the present invention is selected from amantadine sulphate, rimantadine sulphate, memantine sulphate, 3-amino-1-amantadol sulphate, 1-adamantanemethylamine sulphate, N-methyl-1-adamantylamine sulphate, 2-adamantylamine sulphate, 2-methyladamantan-2-amine sulphate, and 2-ethyladamantan-2-amine sulphate. In further embodiments, the pharmaceutically (or veterinary) acceptable salt of the compounds envisaged in the context of the present invention is selected from amantadine sulphate, memantine sulphate, 2-adamantylamine sulphate, 2-methyladamantan-2-amine sulphate, and 2-ethyladamantan-2-amine sulphate.

In certain embodiments, the pharmaceutically (or veterinary) acceptable salt is selected from amantadine sulphate, memantine sulphate, 3-amino-1-amantadol sulphate, 1-adamantanemethylamine sulphate, N-methyl-1-adamantylamine sulphate and 2-adamantylamine sulphate. In further embodiments, the pharmaceutically (or veterinary) acceptable salt is selected from amantadine sulphate, memantine sulphate and 2-adamantylamine sulphate.

In certain embodiments, the present invention envisages the prevention and/or treatment of parvovirus infections in animals and humans by administering combinations of the compounds disclosed herein and other active ingredients. Such combinations may be administered simultaneously or separately. In particular embodiments, the invention provides formulations in which the compounds described herein are combined with other active ingredients, such as non-steroidal anti-inflammatory drugs, interferons, G-CSFs etc. In particular embodiments, the invention provides a compound of the amantadine family, or a solvate, pharmaceutically or veterinary acceptable salt thereof, in admixture with one or more other active ingredients, for use in a method for the treatment or prevention of a parvovirus infection, and/or for lowering parvovirus viral load.

The inventors have moreover surprisingly found that the compounds of the amantadine family described herein are particularly effective when the parvovirus is a virus of the subfamily Parvovirinae. Accordingly, in particular embodiments, the present invention relates to the use of (compositions comprising) one or more compounds of the amantadine family in the treatment or prevention of a parvovirus infection which is caused by a virus of the subfamily Parvovirinae, such as MVM, FPV, CPV-2 and parvovirus B19. More particularly, in certain embodiments, the present invention relates to the (compositions comprising) one or more compounds of the amantadine family for use in a method for lowering the viral load of a virus of the subfamily Parvovirinae and/or methods for lowering the viral load of a virus of the subfamily Parvovirinae comprising administering to a subject in need thereof, an effective amount of (compositions comprising) one or more compounds of the amantadine family so as to reduce the viral load of said virus.

Indeed, the inventors have found that the envisaged compounds are effective against human parvovirus B19, which is the only member of the Parvoviridae family known to be pathogenic in humans. Accordingly, in certain embodiments, the present invention provides in the use of (compositions comprising) a compound of the family of amantadine in the treatment or prevention of a human parvovirus B19 infection in a human. Children are typically more susceptible to parvovirus infection than adults. Parvovirus infection during pregnancy may lead to fetal death. In certain embodiments, the invention provides for the treatment of pregnant women and/or children, more preferably for the treatment and prevention of a parvovirus infection, more preferably an infection with a human parvovirus B19 in a child of an age above 1 year. For treatment of parvovirus infections in a pregnant woman, the compound according to the present invention is preferably memantine or memantine hydrochloride. For treatment of parvovirus infections in a child, the compound according to the present invention is preferably memantine, rimantadine, memantine hydrochloride or rimantadine hydrochloride, more preferably memantine or memantine hydrochloride.

In humans, parvovirus infection may have various expressions such as erythema infectiosum (also known as “fifth disease”), (seronegative) arthritis and anemia. Accordingly, in certain embodiments, the present method envisages the use of a (composition comprising a) compound of the amantadine family in the treatment of one or more symptoms selected from erythema infectiosum, arthritis and anemia.

The invention further relates to the use of (a composition comprising) a compound of the amantadine family in the treatment or prevention of a parvovirus infection in an animal, and/or for the reduction of parvovirus viral load in an animal. Preferably, the animal is a warm-blooded animal. In certain embodiments, said animal is a carnivorous animal. In particular embodiments, said animal is a companion animal such as a canine or a felid or a laboratory animal such as a mouse.

In particular embodiments, the compound according to the present invention are envisaged for use in a method for the treatment or prevention of a canine parvovirus infection in a canine, and/or for the reduction of parvovirus viral load in a canine. In certain embodiments, said canine parvovirus infection is caused by a virus selected from canine parvovirus type 2 (CPV-2) and canine minute virus (CPV-1). In certain embodiments, the CPV-2 virus is CPV-2a, CPV-2b or CPV-2c. In particular embodiments, said canine is a dog. Mortality rates of CPV-2 infected dogs are particularly high in pups. Accordingly, particular embodiments of the invention relate to the treatment or prevention of parvovirus infections in puppies.

The compounds and compositions comprising the compounds described herein are further envisaged for use in the treatment or prevention of a feline panleukopenia virus (FPV) infection in a felid, and/or for the reduction of FPV viral load in a felid. In particular embodiments, the felid is a cat. Although FPV affects cats of all ages, kittens are most susceptible. Accordingly, in further embodiments, the cat is a kitten.

In particular embodiments, the invention relates to the treatment and prevention of a Minute Virus of Mice (MVM) in a rodent, and/or for the reduction of MVM viral load in a rodent such as a mouse, rat or hamster. MVM infections are a common problem in laboratory mice breeding. Therefore, in preferred embodiments, the rodent is a mouse. As will be apparent from the above, the invention also envisages the prevention and treatment of parvovirus infections, and/or the reduction of parvovirus viral load, using one or more compounds of the amantadine family provided in a composition. Accordingly, the present invention provides a composition comprising one or more compounds of the amantadine family, or a solvate, pharmaceutically or veterinary acceptable salt thereof, for use in a method for the treatment or prevention of a parvovirus infection, and/or for use in a method for the reduction of parvovirus viral load. The composition may comprise two or more compounds of the amantadine family, or a solvate, pharmaceutically or veterinary acceptable salt thereof. In particular embodiments, the composition comprises two or more compounds selected from amantadine, rimantadine, memantine, 3-amino-1-amantadol, 1-adamantanemethylamine, N-methyl-1-adamantylamine, 2-adamantylamine, 2-methyladamantan-2-amine, and 2-ethyladamantan-2-amine, or a pharmaceutically or veterinary acceptable salt thereof. In certain embodiments, the composition comprises two or more compounds selected from amantadine, memantine, 2-adamantylamine, 2-methyladamantan-2-amine, and 2-ethyladamantan-2-amine, or a pharmaceutically or veterinary acceptable salt thereof. In specific embodiments, the composition comprises two or more compounds selected from amantadine, memantine, 3-amino-1-amantadol, 1-adamantanemethylamine, N-methyl-1-adamantylamine and 2-adamantylamine or a pharmaceutically or veterinary acceptable salt thereof. In further embodiments, the composition comprises two or more compounds selected from amantadine, memantine and 2-adamantylamine or a pharmaceutically or veterinary acceptable salt thereof.

Typically, the compositions used in the treatment and/or prevention of a parvovirus infection as described herein will comprise in addition to one or more compounds from the amantadine family, and optionally other active ingredients, one or more pharmaceutically or veterinary acceptable carriers or excipients. Such compositions are typically also referred to as pharmaceutical compositions.

The present invention also encompasses pharmaceutical composition comprising one or more compounds of the amantadine family, or a solvate, pharmaceutically acceptable salt or veterinary acceptable salt thereof, for use in a method for the treatment or prevention of a parvovirus infection in a human or a warm-blooded animal, and/or for use in a method for the reduction of the parvovirus viral load in a warm-blooded animal.

The term “pharmaceutically acceptable carrier” or “veterinary acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier or veterinary acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable carriers are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol, benzyl alcohol), isotonic agents (such as sugars or sodium chloride) and the like, provided the same are consistent with pharmaceutical practice, i.e. carriers and additives which do not create permanent damage to mammals. The compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents may also be prepared by micronisation, for instance in view to obtain them in the form of microspheres usually having a diameter of about 1 to 10 μm, namely for the manufacture of microcapsules for controlled or sustained release of the active ingredients.

The present invention also encompasses pharmaceutical composition comprising one or more compounds of formula (I), or a stereoisomer, salt, solvate or hydrate thereof, for use in a method for the treatment or prevention of a parvovirus infection in a human or a warm-blooded animal.

In some embodiments, the pharmaceutical composition comprises one or more compounds selected from amantadine, rimantadine, memantine, 3-amino-1-amantadol, 1-adamantanemethylamine, N-methyl-1-adamantylamine, 2-adamantylamine2-methyladamantan-2-amine, and 2-ethyladamantan-2-amine. In certain embodiments, the pharmaceutical composition comprises one or more compounds selected from amantadine, memantine, 2-adamantylamine, 2-methyladamantan-2-amine, and 2-ethyladamantan-2-amine. In some embodiments, the pharmaceutical composition comprises one or more compounds selected from amantadine, rimantadine, memantine, 3-amino-1-amantadol, 1-adamantanemethylamine, N-methyl-1-adamantylamine or 2-adamantylamine, for example amantadine or memantine or combination thereof.

The present invention provides methods for the treatment and/or prevention of a parvovirus infection in an animal or human, and/or for the reduction of parvovirus viral load in an animal or human. Typically the methods of treatment encompass administration of the compounds or compositions described herein to a human or animal suffering from a parvovirus infection. The animal or human may have been diagnosed as suffering from a parvovirus infection or may be identified as having parvovirus associated symptoms. In particular embodiments, the methods are envisaged for the treatment of human patients identified has having B19 parvovirus associated symptoms and/or patients identified as infected or carrying B19 parvovirus or characterized by the presence of antibodies to B19 parvovirus. Similarly, in particular embodiments, the methods are envisaged for the treatment of animal of a felid suffering from feline panleukopenia virus infection. In particular embodiments, the methods are envisaged for the treatment of felids identified has having panleukopenia virus infection associated symptoms and/or felids identified as infected or carrying panleukopenia virus or characterized by the presence of antibodies to panleukopenia virus. Typically the methods of treatment involve the administration of an effective amount of the compounds or compositions described herein.

For the methods of prevention, these typically encompass administration of the compounds or compositions described herein to a human or animal susceptible to a parvovirus infection. Such susceptibility can be environmental, genetic or related to other factors such as age. The optimal dose and administration frequency of the (compositions comprising a) compound of the amantadine family in the methods for treatment or prevention of a parvovirus infection may depend on the severity of the infection and characteristics of the patient, such as age and/or weight. In particular embodiments, the compound or compositions may be administered once or twice per day, for at least 3, 4, 5, 6 or 7 days. For humans, a dose between 50 and 600 mg per day, more preferably between 100 and 300 mg per day, is considered suitable.

The formulation of the compositions envisaged in the context of the present invention will be determined by the mode of administration and the dosage envisaged.

For buccal administration, the composition can be in the form of tablets formulated in conventional manner. For example, tablets and capsules for oral administration can contain conventional excipients such as binding agents (e.g., syrup, acacia, gelatin, sorbitol, tragacanth, mucilage of starch or polyvinylpyrrolidone), fillers (e.g., lactose, sugar, microcrystalline cellulose, maize-starch, calcium phosphate or sorbitol), lubricants (e.g., magnesium stearate, stearic acid, talc, polyethylene glycol or silica), disintegrants (e.g., potato starch or sodium starch glycolate), or wetting agents (e.g., sodium lauryl sulfate). Tablets may be coated according to methods well known in the art.

Alternatively, the composition described herein can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; non-aqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions described herein may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds described herein can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Additionally, compositions described herein can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

In line with the above, the present invention also provides the use of (a composition comprising) one or more compounds selected from the amantadine family such as but not limited to amantadine, rimantadine, memantine, 3-aminol-amantadol, 1-adamantanemethylamine, N-methyl-1-adamantylamine, 2-adamantylamine, 2-methyladamantan-2-amine, and 2-ethyladamantan-2-amine, or a solvate or pharmaceutically or veterinary acceptable salt thereof, for the manufacture of a medicament for the treatment or prevention of a parvovirus infection in a human or a warm-blooded animal.

The following examples are provided for the purpose of illustrating the present invention and by no means are meant and in no way should be interpreted to limit the scope of the present invention.

### EXAMPLES

### Example 1

### Inhibition of Feline Panleukopenia Virus and Canine Parvovirus 2c Replication by Amantadine, Amantadine Hydrochloride (HCl), 2-Adamantylamine HCl, 3-Amino-1-Amantadol, N-Methyl-1-Adamantylamine, 1-Adamantanemethylamine, Rimantadine and Memantine in Cell Culture

### Materials and Methods

Amantadine, amantadine HCl, 2-adamantylamine HCl, 3-amino-1-amantadol, N-methyl-1-adamantylamine, 1-adamantanemethylamine, rimantadine and memantine were dissolved in Dulbecco's phosphate buffered saline (DPBS) at a concentration of 10 mM. Crandell Reese Feline Kidney (CrFK) cells were grown in Dulbecco minimum essential medium (DMEM, Life Technologies) containing 1% sodium bicarbonate (Life Technologies), 1% L-Glutamine (Life Technologies) and 5% fetal calf serum (FCS, Biochrom). In a 96 well plate a serial dilution of compound was added together with 10 TCID₅₀ of feline panleukopenia virus (FPV) or 10 TCID₅₀ canine parvovirus 2c (CPV-2c) and 20,000 CrFK cells. Infected cells without any added compound were used as positive controls (virus control) and uninfected cells were used as negative controls (cell control). The cells were incubated for 4 days and viral cytopathogenic effect (CPE) was scored visually. EC₅₀-values were determined as the concentration of compound that inhibited 50% of the viral replication as compared with virus control.

### Results

The concentrations of compound that effectively inhibit 50% of the FPV or CPV-2c replication as measured by CPE visual scoring assay are given in Table 1. Amantadine, amantadine HCl, memantine and 2-adamantylamine HCl were found to be most effective.

TABLE 1
EC₅₀ values expressed in μM of amantadine, amantadine
hydrochloride (HCl), 2-adamantylamine HCl, 3-amino-1-amantadol,
N-methyl-1-adamantylamine, 1-adamantanemethylamine, rimantadine
and memantine against FPV and CPV-2c as measured by the CPE
visual scoring assay.

| | EC₅₀-value | EC₅₀-value |
|---|---|---|
| Compound | (μM) for FPV | (μM) for CPV-2c |
| Amantadine | 18.73 | 22.61 |
| Amantadine HCl | 33.34 | 14.01 |
| 2-adamantylamine HCl | 17.34 | 5.12 |
| Rimantadine | >250 | >250 |
| 3-amino-1-amantadol | >250 | >250 |
| N-methyl-1-adamantylamine | >250 | >250 |
| 1-adamantanemethylamine | >250 | >250 |
| Memantine | 23.33 | 23.87 |

### Example 2

### Inhibition of Feline Panleukopenia Virus and Canine Parvovirus 2c Production and Viral DNA Production in Cell Culture by Amantadine, Amantadine Hydrochloride (HCl), and Memantine

### Materials and Methods

Amantadine, amantadine hydrochloride (HCl) and memantine were dissolved in Dulbecco's phosphate buffered saline (DPBS) at a concentration of 10 mM. Crandell Reese Feline Kidney (CrFK) cells were grown in Dulbecco minimum essential medium (DMEM, Life Technologies) containing 1% sodium bicarbonate (Life Technologies), 1% L-Glutamine (Life Technologies) and 5% fetal calf serum (FCS, Biochrom). In a 24 well plate 100,000 CrFK cells were seeded on day 0. On day 1, the medium was removed and 10 TCID₅₀ of FPV or 10 TCID₅₀ CPV-2c was added and incubated for 2 h at 37° C. in humidified conditions. After incubation, medium was removed and CrFK cells were washed with DPBS. Subsequently, a serial dilution of compound in culture medium was added (250, 50, and 10 μM). Infected cells without any added compound were used as positive controls (virus control) and uninfected cells were used as negative controls (cell control). The cells were incubated at 37° C. in humidified conditions. After 4 days of incubation medium containing newly produced virus particles was collected and viral titers were determined using CrFK cells. In addition, 200 μL of supernatant was extracted to obtain FPV or CPV-2c DNA using the QIAamp DNA extraction kit (Qiagen). The extracted FPV DNA was detected by a real time PCR using the Taqman Fast Universal mastermix (Life Technologies) and the standard Fast cycling protocol using the StepOnePlus real time device (Applied Biosystems). In the PCR mixture an optimized 900 nM of each forward and reverse primer was used and 250 nM FAM-BHQ1 labeled probe as published by Decaro et al. (J. Virol. Methods 2008, 147, 67-71). EC₅₀ values were determined as the concentration of compound that inhibited 50% of the viral DNA.

### Results

The concentration of amantadine, amantadine hydrochloride (HCl) and memantine that effectively inhibits 50% of FPV and CPV-2c DNA production (EC₅₀) as measured by quantitative PCR is given in Table 2. Amantadine HCl was found to be the most effective in viral DNA reduction.

Reduction of CPV-2c and FPV titers under increasing concentrations of amantadine hydrochloride (HCl) and memantine are shown in Table 3a and 3b, respectively. Viral titers are determined by titration of the produced virus on CrFK cells and incubated for 5 days. The obtained titers where compared with the control (virus production without addition of compound). Amantadine HCl was found to be the most efficient in reduction of FPV and CPV-2c production.

TABLE 2
EC₅₀ values expressed in μM of FPV and CPV-2c DNA reduction by the
compounds as measured by quantitative PCR

| Compound | EC₅₀-value (μM) for FPV | EC₅₀-value (μM) for CPV-2c |
|---|---|---|
| Amantadine | 57.15 | 53.46 |
| Amantadine | 30.14 | 6.59 |
| HCl | | |
| Memantine | 42.58 | 17.89 |

TABLE 3a
Titer reduction of CPV-2c under different concentrations of amantadine,
amantadine hydrochloride and memantine. Data presented as log10
reduction compared with control (virus production without addition
of compound).

| | Concentration (μM) | | |
|---|---|---|---|
| Compound | 250 | 50 | 10 |
| Amantadine | 2.23 | 2.23 | 0 |
| Amantadine HCl | 2.71 | 2.71 | 0.50 |
| Memantine | 1.55 | 0.50 | 0 |

TABLE 3b
Titer reduction of FPV under different concentrations of amantadine,
amantadine hydrochloride and memantine. Data presented as log10
reduction compared with control (virus production without addition
of compound).

| | Concentration (μM) | | |
|---|---|---|---|
| Compound | 250 | 50 | 10 |
| Amantadine | 1.43 | 0.48 | 0 |
| Amantadine HCl | 2.44 | 1.50 | 0 |
| Memantine | 1.41 | 0.71 | 0 |

### Example 3

### Inhibition of Feline Panleukopenia Virus and Canine Parvovirus 2c Production and Viral DNA Production in Cell Culture by 2-Methyladamantan-2-Amine Hydrochloride and 2-Ethyladamantan-2-Amine Hydrochloride

2-methyladamantan-2-amine HCl and 2-ethyladamantan-2-amine HCl were dissolved in Dulbecco's phosphate buffered saline (DPBS) at a concentration of 10 mM. The inhibition experiments and the preparation thereof were performed as described above for Example 2.

The concentration of 2-methyladamantan-2-amine HCl and 2-ethyladamantan-2-amine HCl that effectively inhibits 50% of FPV and CPV-2c virus and DNA production (EC₅₀) as measured by a cytopathic effect (CPE) visual scoring assay and by quantitative PCR is given in Table 4a and Table 4b, respectively. 2-methyladamantan-2-amine HCl and 2-ethyladamantan-2-amine HCl were both found to be very effective in viral DNA reduction.

TABLE 4a
EC₅₀ values expressed in μM for antiviral activity of the compounds
against FPV and CPV-2c as measured by the CPE visual scoring assay.

| | EC₅₀-value | EC₅₀-value (μM) |
|---|---|---|
| Compound | (μM) for FPV | for CPV-2c |
| 2-methyladamantan-2-amine | 1.95 | 2.25 |
| 2-ethyladamantan-2-amine | 2.42 | 4.20 |

TABLE 4b
EC₅₀ values expressed in μM for FPV and CPV-2c DNA reduction by the
compounds as measured by quantitative PCR

| | EC₅₀-value | EC₅₀-value (μM) |
|---|---|---|
| Compound | (μM) for FPV | for CPV-2c |
| 2-methyladamantan-2-amine | <0.781 | 1.14 |
| 2-ethyladamantan-2-amine | 1.31 | 2.44 |

### Example 4

### Treatment of Mice Infected with Minute Virus of Mice with Amantadine Hydrochloride

Severe combined immunodeficient (SCID) mice were infected with a preparation of Minute Virus of Mice type p (MVMp) by intraperitoneal inoculation. Whereas MVMp is not lethal to healthy mice, administration of high titers of MVMp resulted in high mortality rates in SCID mice as shown in Table 5. However, treatment of the mice from day 0 of infection onwards with 10 mg/kg amantadine HCl resulted in a significant survival of the mice.

TABLE 5
Mortality rate of MVMp infected mice of amantadine HCl-treated and
untreated mice after 2 weeks.

| Compound | Mortality rate after 2 weeks |
|---|---|
| control (untreated) | 82% |
| Amantadine HCl-treated | 16% |

### Example 5

### Treatment with Amantadine Hydrochloride of Dogs Naturally Infected with Canine Parvovirus Type 2

A beagle puppy of 2 months of age with clear clinical signs of parvovirus infection including enteritis, severe bloody diarrhea, vomiting and fever, and PCR-positive for CPV2 was treated for 2 weeks twice daily with 10 mg/kg amantadine HCl. Unexpectedly, clinical signs improved from day 3 onwards and the dog recovered completely after 2 weeks of treatment.

1. A method for treating a parvovirus infection in a human or a warm-blooded animal, the method comprising administering a compound of formula (I), or a stereoisomer, salt, solvate, or hydrate thereof; 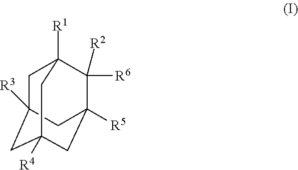
wherein R¹ is amino and R² and R⁶ are hydrogen; or wherein R¹ is hydrogen, R² is selected from amino, aminoC₁₋₆alkyl or C₁₋₆alkylamino, and R⁶ is hydrogen or C₁₋₆alkyl;
and
R³, R⁴ and R⁵ are each independently selected from hydrogen and C₁₋₆alkyl to the human or a warm-blooded animal having the parvovirus infection.

2. The method according to claim 1, wherein said compound is selected from amantadine, memantine, 2-adamantylamine, 2-methyladamantan-2-amine, and 2-ethyladamantan-2-amine, or a solvate, hydrate, pharmaceutically acceptable salt or veterinary acceptable salt thereof.

3. The method according to claim 1, wherein R⁶ is hydrogen.

4. The method according to claim 3, wherein said compound of the amantadine family is a compound of formula (Ia), or a stereoisomer, salt, solvate or hydrate thereof; 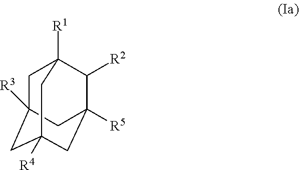
wherein R¹ is hydrogen and R² is selected from amino, aminoC₁₋₆alkyl or C₁₋₆alkylamino; and
R³, R⁴ and R⁵ are each independently selected from hydrogen and C₁₋₆alkyl.

5. The method according to claim 3, wherein said compound is selected from memantine and 2-adamantylamine, or a solvate, hydrate, pharmaceutically acceptable salt or veterinary acceptable salt thereof.

6. The method according to claim 1, wherein R¹ is hydrogen, R² is selected from amino, aminoC₁₋₆alkyl or C₁₋₆alkylamino, and R⁶ is hydrogen or C₁₋₆alkyl.

7. The method according to claim 6, wherein said compound is 2-methyladamantan-2-amine or 2-ethyladamantan-2-amine; or a solvate, hydrate, pharmaceutically acceptable salt or veterinary acceptable salt thereof.

8. The method according to claim 1, wherein said parvovirus infection is caused by a virus of the subfamily Parvovirinae.

9. The method according to claim 1, wherein the infection is human parvovirus B19 infection in a human.

10. The method according to claim 1, wherein said infection is in a canine.

11. The method according to claim 10, wherein the infection is a canine parvovirus infection in a canine.

12. The method according to claim 1, wherein said infection is in a felid.

13. The method according to claim 12, wherein the infection is a feline panleukopenia virus infection in a felid.

14. The method according to claim 1, wherein the viral load of said parvovirus is lowered as a result of the method.

15. A method for treating a parvovirus infection in a human or a warm-blooded animal, the method comprising administering a pharmaceutical composition comprising one or more compounds of formula (I), or a solvate, pharmaceutically acceptable salt or veterinary acceptable salt thereof; 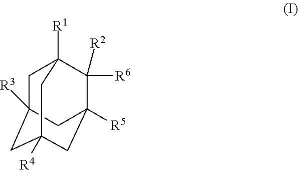
wherein R¹ is amino and R² and R⁶ are hydrogen; or wherein R¹ is hydrogen, R² is selected from amino, aminoC₁₋₆alkyl or C₁₋₆alkylamino, and R⁶ is hydrogen or C₁₋₆alkyl;
and
R³, R⁴ and R⁵ are each independently selected from hydrogen and C₁₋₆alkyl;
to the human or a warm-blooded animal having the parvovirus infection.
